(19) United States
(12) Patent Application Publication
(10) Pub. No.: US 2004/0044224 A1
(43) Pub. Date: Mar. 4, 2004

(54) Process for preparing 2-phenyl-3-aminobenzothiophenes

(76) Inventors: Preston Charles Conrad, West Lafayette, IN (US); John Paul Gardner, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/344,246

(22) PCT Filed: Aug. 21, 2001
(86) PCT No.: PCT/US01/22608

(51) Int. Cl. C07D 333/72

(52) U.S. Cl. 549/6; 549/55

(57) ABSTRACT

The present invention relates to a one pot process for preparing a compound of formula I: I; from a compound of formula II and III: I; III.

### BACKGROUND OF THE INVENTION

Previously, Beck (Beck, J. R., J. Het. Chem., 15:513, 1979) had developed a two-step process for preparing 2-aryl-3-aminobenzo[b]thiophenes. Beck's first step involved coupling a benzylthiol to an ortho-nitrobenzonitrile using potassium hydroxide in dimethylformamide. Beck further taught that this coupled product could subsequently be cyclized to a 2-aryl-3-aminobenzo[b]thiophene in a benzene solution of potassium t-butoxide. These transformations from Beck are summarized pictorially below:

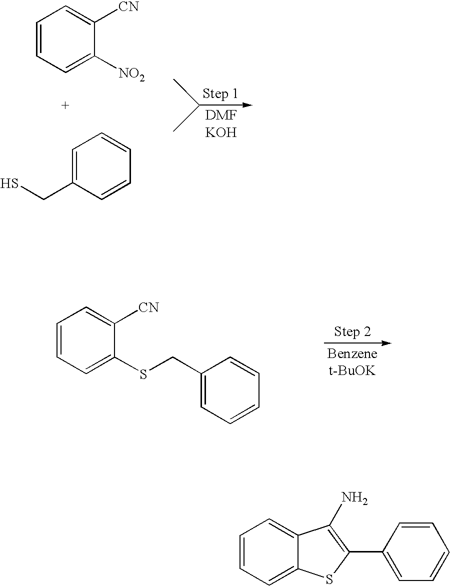

If conditions similar to those employed for Step 1 were used in an attempt to effect the cyclization of Step 2, said conditions would not be expected to afford the desired product. Additionally, when conditions similar to those employed for Step 2 were used by Applicants to couple a thiol to a derivatized benzonitrile, said conditions afforded substantial competition between an undesired inter-molecular reaction and the desired intra-molecular reaction.

As a result, the chemistry disclosed by Beck is not amenable to performing the above chemical transformations in a one pot reaction and is, therefore, less amenable to large-scale manufacturing.

It would be highly desired and advantageous to be able to accomplish the overall transformation above in a one pot chemical reaction.

### BRIEF SUMMARY OF THE INVENTION

The present invention relates to a process for preparing a compound of formula I:

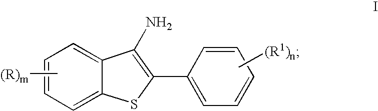

wherein:

m is 0 or 1;

R and R¹ are independently at each occurrence OH, NH₂, CF₃, CCl₃, CN, halo, C₁-C₆ alkoxy, C₃-C₇ cycloalkoxy, OC(O)(C₁-C₆ alkyl), OC(O)(C₃-C₇ cycloalkyl), OCO₂(C₁-C₆ alkyl), OCO₂(C₃-C₇ cycloalkyl), OSO₂(C₄-C₆ alkyl), OCON(R²)₂, OAr, OCOAr, OCO₂Ar, OCH₂Ar, OC(O)CH₂Ar, OCO₂CH₂Ar, OPO(C₁-C₆ alkyl)₂, OPO(Ar)₂, OPO(C₁-C₆ alkoxy)₂, OPO(OAr)₂, OCH₂NHC(O)R³, OCH₂OR³, OCH₂SR³;

R² is independently at each occurrence hydrogen, C₁-C₆ alkyl, C₃-C₇ cycloalkyl, or Ar;

R³ is C₁-C₆ alkyl or Ar; and

Ar is an optionally substituted phenyl group;

which includes reacting a compound of formula II:

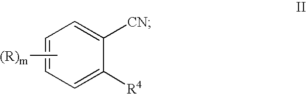

where R⁴ is fluoro or nitro; with a compound of formula III:

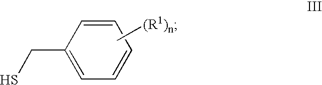

in the presence of a suitable polar aprotic solvent and between 1.01 and 3.0 equivalents, relative to the formula III compound, of a suitable kinetic base.

### DETAILED DESCRIPTION OF THE INVENTION

The compounds of formula I and their derivatives are benzo[b]thiophenes hereafter referred to simply as benzothiophenes. The following numbering system for the substituents around the benzothiophene ring is employed throughout.

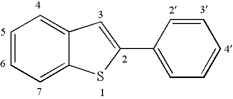

General terms used in the description of chemical formulas bear their usual meanings. For example, the term “C₁-C₆ alkyl” refers to a straight or branched aliphatic alkyl chain of 1 to 6 carbon atoms including, methyl, ethyl, propyl, isopropyl, n-butyl, s-butyl, t-butyl, pentyl, n-hexyl, and the like. The term “C₄-C₆ alkyl” refers to a n-butyl, n-pentyl, and n-hexyl group. The term “C₃-C₇ cycloalkyl” refers to a cyclopropyl, cyclobutyl, cyclopentyl, cyclohexyl, and cycloheptyl group.

The terms “C₁-C₆ alkoxy” and “C₃-C₇ cycloalkoxy” refer to a C₁-C₆ alkyl group and C₃-C₇ cycloalkyl group, respectively, attached through an oxygen.

The terms “halo” and “halogen” are used interchangeably herein and refer to fluoro, chloro, bromo, and iodo.

The term “suitable kinetic base” refers to a base that provides a non-reversible deprotonation of a compound of formula III's thiol hydrogen. More specifically, a suitable kinetic base is a base whose pKa (measured in dimethylsulfoxide) is at least 35. Examples of suitable kinetic bases include alkyl metals (for example, n-butyl lithium, s-butyl lithium, and t-butyl lithium or ethyl magnesium bromide and the like), metal amides such as lithium diisopropyl amide, potassium, lithium, or sodium salts of dimethylsulfoxide or hexamethydisilazane, or metal hydrides (for example, sodium, lithium, or potassium hydride).

The term “suitable aprotic polar solvent” refers to any aprotic polar solvent, or mixture of solvents resulting in a aprotic polar mixture, inert to the ongoing reaction that sufficiently solubilizes the reactants to afford a medium within which to effect the desired reaction. Suitable solvents include methylene chloride, chloroform, 1,2-dichloroethane, diethyl ether, acetonitrile, ethyl acetate, 1,3-dimethyl-2-imidazolidinone, tetrahydrofuran, dimethylformamide, toluene, chlorobenzene, dimethylsulfoxide, N-methylpyrrolidinone, dimethylacetamide, hexamethylphosphoramide, mixtures thereof, and the like.

An “optionally substituted phenyl group” is a phenyl group that is substituted 0-2 times with a moiety independently selected from the group consisting of: C₁-C₄ alkyl, C₁-C₄ alkoxy, hydroxy, nitro, chloro, fluoro and tri(chloro or fluoro)methyl.

The term “nucleophilic source of a halogen” refers to halogenating reagents such as benzeneseleninylchloride/aluminum chloride, thionyl chloride, CsSO₄F, NFTh, N-bromo succinimide, N-chloro succinimide, N-iodo succinimide, molecular bromine, molecular iodine, and the like.

The term “acid addition salt” is meant to include, but not be limited to, the sulfate, pyrosulfate, bisulfate, sulfite, bisulfite, phosphate, mono-hydrogenphosphate, dihydrogenphosphate, metaphosphate, pyrophosphate, chloride, bromide, iodide, acetate, propionate, decanoate, caprylate, acrylate, formate, isobutyrate, heptanoate, propiolate, oxalate, malonate, succinate, suberate, sebacate, fumarate, maleate, 2-butyne-1,4 dioate, 3-hexyne-2, 5-dioate, benzoate, chlorobenzoate, hydroxybenzoate, methoxybenzoate, phthalate, xylenesulfonate, phenylacetate, phenylpropionate, phenylbutyrate, citrate, lactate, hippurate, β-hydroxybutyrate, glycollate, maleate, tartrate, methanesulfonate, propanesulfonate, naphthalene-1-sulfonate, naphthalene-2-sulfonate, mandelate and like salts. A preferred acid addition salt is the hydrochloride salt.

The novel process of the present invention is illustrated in Scheme 1 below where m, n, R, R¹, and R⁴ are as defined above.

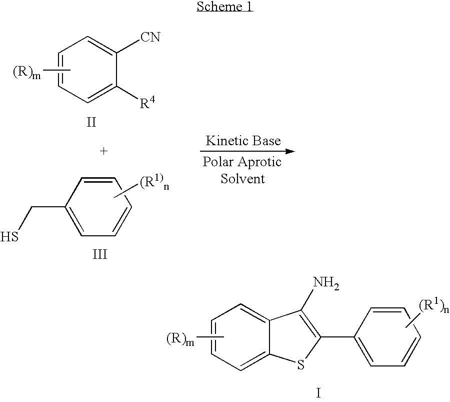

A compound of formula III may be added to a solution or suspension of a kinetic base, preferably sodium hydride, in a suitable polar aprotic solvent, preferably dimethylformamide, to effect deprotonation of the thiol hydrogen. A formula II compound may then be added to the reaction followed by stirring/agitation of the resulting mixture for a time sufficient to complete the coupling/cyclization. The resulting compound of formula I may be isolated by standard techniques. For example, the addition of water to the reaction mixture typically results in the precipitation of the formula I compound which may be isolated by vacuum filtration.

In general, the deprotonation is performed cold, usually by cooling the solution/suspension of the kinetic base with an ice/water bath and by adding the formula III compound at a rate sufficient to maintain temperatures around 15° C. or less. Once the addition of the formula III compound is complete, the mixture is typically allowed to warm to room temperature prior to addition of the formula II compound.

Typically, a molar excess of kinetic base (from 1.01 to about 3 equivalents), relative to the compound of formula III, is used. More typically, 2.0 to about 3.0 equivalents are employed while most typically, 2.0 to about 2.5 equivalents are employed. A molar excess of between 2.0 and 2.15 is most preferred. A slight molar excess of a compound of formula III (1.01 to about 1.5 equivalents), relative to the compound of formula II, is typically used. More typically, 1.05 to about 1.25 equivalents are employed while most typically, 1.1 to about 1.2 equivalents are employed.

Preferred compounds of formula II for use in the present process are those where m is 1. Of these preferred compounds, even more preferred are those compounds of formula II where R is benzyloxy, methoxy, isopropoxy, trifluoromethyl, cyano, chloro, or amino. Of these preferred compounds, the most preferred are those where R is methoxy, isopropoxy or benzyloxy and is at the meta-position relative to R⁴. Preferred compounds of formula III for use in the present process are those where n is 1. Of these preferred compounds, even more preferred are those compounds of formula III where R¹ is benzyloxy, methoxy, isopropoxy, trifluoromethyl, cyano, chloro, or amino. Of these preferred compounds, the most preferred are those where R¹ is methoxy, isopropoxy or benzyloxy and is at the para-position. Thus, preferred products of the above reaction include, but are not limited to, 2-(4-methoxyphenyl)-3-amino-6-methoxybenzothiophene, 2-(4-isopropoxyphenyl)-3-amino-6-methoxybenzothiophene, and 2-(4-benzyloxyphenyl)-3-amino-6-methoxybenzothiophene.

In another embodiment of the present invention, the diazonium salt (formula IV compound) of a compound of formula I may be prepared. This salt can be reduced to afford the corresponding 3-hydrido compound of formula V or can be reacted with a nucleophilic source of a halogen to prepare the corresponding 3-halo compound of formula VI as illustrated in Scheme 2 below where n, m, R, R¹ are as defined above.

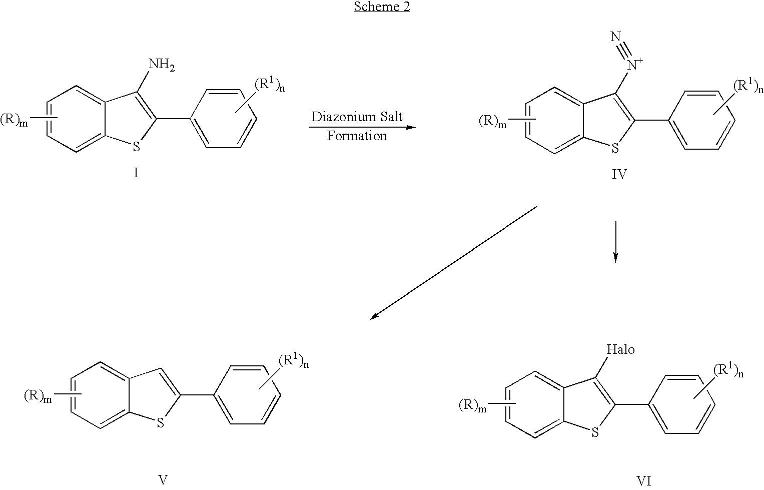

Methods for preparing diazonium salts from amino compounds, for reducing a diazonium salt to the corresponding hydrido compound, or for converting a diazonium salt to the corresponding bromo compound (Sandmeyer Reaction) are well known generally in the art. Specific examples of these transformations are provided in the Examples section below.

Preferred compounds of formula IV are those produced from the preferred compounds of formula I. A particularly preferred compound of formula IV for use in the reduction reaction is one where m and n are 1, R is 6-methoxy, and R¹ is 4′-methoxy. A particularly preferred compound of formula V, therefore, is 2-(4-methoxyphenyl)-6-methoxybenzo[b]thiophene. A particularly preferred compound of formula IV for use in the halogenation reaction is one where m and n are 1, R is 6-isopropoxy or 6-benzyl, and R¹ is 4′-methoxy. Preferred compounds of formula VI, therefore, include 2-(4-methoxyphenyl)-3-bromo-6-isopropoxybenzo[b]thiopnene and 2-(4-methoxyphenyl)-3-bromo-6-benzyloxybenzo[b]thiophene.

In another embodiment, a benzothiophene of formula V may be acylated with a compound of formula VII, optionally deprotected and optionally salified to form a compound of formula VIII, or a pharmaceutical salt thereof, as illustrated in Scheme 3 below where p is 0, 1 or 2; R⁵ and R⁶ are independently C₁-C₄ alkyl, or combine together with the nitrogen to which they are attached to form a piperidinyl, pyrrolidinyl, methylpyrrolidinyl, dimethylpyrrolidinyl, morpholino, dimethylamino, diethylamino, or 1-hexamethyleneimino ring; and R⁷ is chloro, bromo or hydroxy.

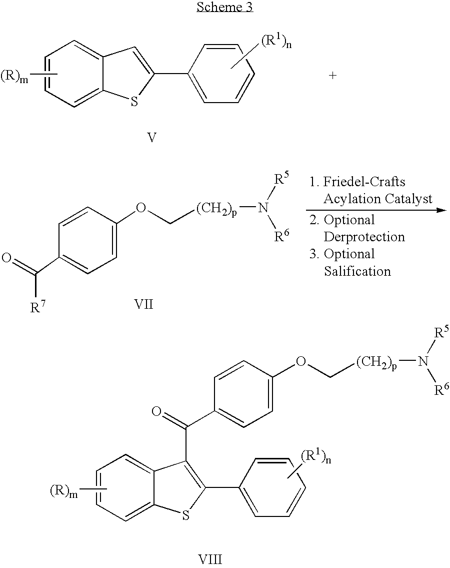

The acylation and optional deprotection and salification reactions may be performed essentially as described in U.S. Pat. Nos. 4,380,635, 4,418,068, 5,629,425 and 5,731,327, the teachings of each are herein incorporated by reference. Preferably, the acylation catalyst is boron trichloride or tribromide and is most preferably boron trichloride. In addition, the hydrochloride or hydrobromide salt of a compound of formula VII is preferably employed in the acylation reaction. When these preferred reactants are employed along with the preferred catalyst, the methyl protecting groups are preferably removed by reaction with additional boron trichloride or tribromide, most preferably with additional boron trichloride (see U.S. Pat. No. 5,629,425 or 5,731,327). A preferred compound of formula VII is the hydrochloride salt of 4-(2-piperidin-1-ylethoxy)benzoyl chloride. A preferred compound of formula VIII then is the hydrochloride salt of 2-(4-hydroxyphenyl)-3-(4-(2-piperidin-1-ylethoxy)benzoyl)-6-hydroxybenzothiophene.

In another preferred embodiment, a compound of formula VI may be oxidized, reacted with a nucleophile of formula IX, reduced, optionally deprotected, and optionally salified to prepare a compound of formula X, or pharmaceutical salt thereof, as illustrated in Scheme 4 below.

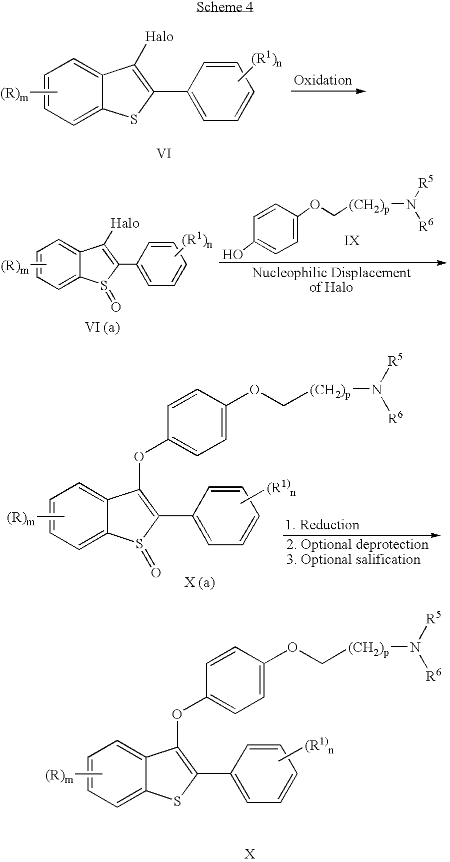

The oxidation, nucleophilic displacement of halo, reduction, and optional deprotection and salification reactions may be performed essentially as described in U.S. Pat. Nos. 5,510,357 and 5,723,474, the teachings of each are herein incorporated by reference.

A preferred compound of formula IX is 4-(2-piperidin-1-ylethoxy)phenol. When the process of Scheme 4 is performed with a preferred compound of formula VI, the deprotection reaction is preferably performed to selectively remove the 6-isopropyl or 6-benzyl protecting group without significantly removing the 4′-methyl group. In addition, the optional salification is also preferably performed in order to prepare the hydrochloride salt of the compound of formula X. Thus, the preparation of the hydrochloride salt of 2-(4-methoxyphenyl)-3-(4-(2-piperidin-1-ylethoxy)phenoxy-6-hydroxybenzo[b]thiophene is a preferred object of the present invention. Methods for carrying out the selective deprotection and salification reactions may be found in the U.S. Pat. No. 5,723,474.

Compounds of formula II and III are known in the art and are generally commercially available or are prepared by methods well known in the art from readily available starting materials.

### EXAMPLES

### Example 1

### 2-(4-Methoxyphenyl)-3-amino-6-benzyloxy-benzo[b]thiophene

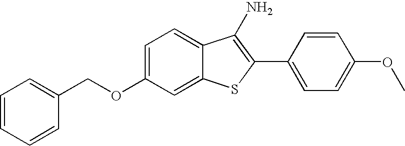

A 100 ml three neck round bottom flask fitted with nitrogen inlet, thermometer, 10 ml addition funnel, nitrogen outlet and magnetic stirrer was charged with 440 mg (11 mmol) of sodium hydride (60% dispersion in mineral oil) and 18 ml of dimethylformamide (DMF) under a nitrogen atmosphere. The suspension was cooled to 0° C. and 0.80 ml of 4-methoxybenzylthiol (5.75 mmol) was added dropwise over 5 minutes. The addition funnel was rinsed with 3 ml of DMF, the cooling bath was removed and the temperature of the mixture was allowed to reach 20° C. over 30 minutes. To the off-white mixture was added 1.44 g (5 mmol) of 2-fluoro-4-(benzyloxy)benzonitrile with 4 ml of DMF rinse. This resulted in a yellow mixture and an exotherm to 26° C. The mixture was stirred at ambient temperature for 2 hours. To the resulting dark orange mixture was added 25 ml of water over 20 minutes causing a precipitate to form. The thick mixture was stirred at room temperature for 1 hour. The solid was collected by filtration and washed with 20 ml of a 1:1 mixture of DMF and water, then 2 times with 10 ml of water, then 2 times with 10 ml of hexane. The solid was dried in a vacuum oven at 45° C. to provide 1.76 g of the title compound. MS: 362.1.

### Example 2

### 2-(4-Methoxyphenyl)-3-amino-benzo[b]thiophene

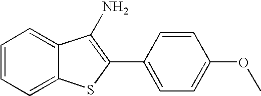

The procedure of Example 1 was repeated using 0.54 ml (5 mmol) of 2-fluorobenzonitrile to prepare 1.26 g of the title compound. MS(ESI+) 256.2.

### Example 3

### 2-(4-Methoxyphenyl)-3-amino-6-trifluoromethyl-benzo[b]thiophene

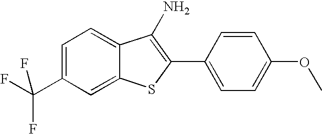

The procedure of Example 1 was repeated using 1.08 g (5 mmol) of 2-nitro-4-(trifluoromethyl)benzonitrile to prepare 1.46 g of the title compound. MS(ESI+) 324.4.

### Example 4

### 2-(4-Methoxyphenyl)-3-amino-4-cyanobenzo[b]thiophene

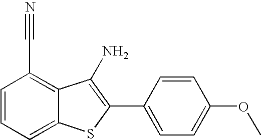

The procedure of Example 1 was repeated using 870 mg (5 mmol) of 3-nitrophthalonitrile to prepare 1.28 g of the title compound. MS(ESI+) 281.1

### Example 5

### 2-(4-Methoxyphenyl)-3-amino-6-chloro-benzo[b]thiophene

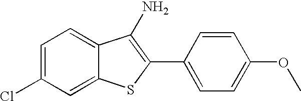

The procedure of Example 1 was repeated using 780 mg (5 mmol) of 4-chloro-2-fluorobenzonitrile to prepare 1.42 g of the title compound. MS(ESI+) 348.2.

### Example 6

### 2-(4-Methoxyphenyl)-3,4-diamino-benzo[b]thiophene

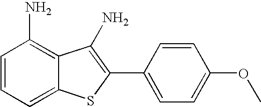

The procedure of Example 1 was repeated using 680 mg (5 mmol) of 2-amino-6-fluorobenzonitrile to prepare 870 mg of the title compound. MS(ESI+) 271.4.

### Example 7

### 2-Phenyl-3-amino-6-benzyloxy-benzo[b]thiophene

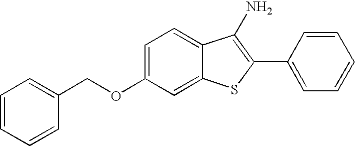

The procedure of Example 1 was repeated using 0.65 ml (5.5 mmol) of benzylmercaptan to prepare 1.60 g of the title compound. MS(ESI+) 332.2.

### Example 8

### 2-(4-Chlorophenyl)-3-amino-6-benzyloxy-benzo[b]thiophene

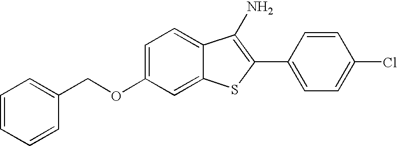

The procedure of Example 1 was repeated using 0.73 ml (5.5 mmol) of 4-chlorobenzenemethanethiol to prepare 1.82 g of the title compound. MS(ESI+) 365.3.

### Example 9

### 2-(3-Trifluoromethylphenyl)-3-amino-6-benzyloxy-benzo[b]thiophene

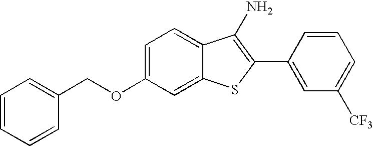

The procedure of Example 1 was repeated using 1.15 g (5.4 mmol) of 3-trifluoromethylbenzenemethanethiol to prepare 1.92 g of the title compound. MS(ESI+) 400.1.

### Example 10

### 2-(4-Fluorophenyl)-3-amino-6-benzyloxy-benzo[b]thiophene

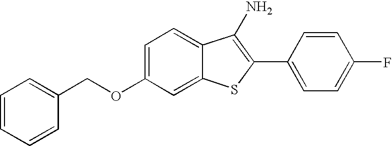

The procedure of Example 1 was repeated using 800 mg (5.46 mmol) of 4-fluorobenzenemethanethiol to prepare 1.69 g of the title compound. MS(ESI+) 349.1.

### Example 11

### 2-Phenyl-3-amino-benzo[b]thiophene

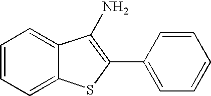

The procedure of Example 1 was repeated using 0.65 ml (5.5 mmol) of benzylmercaptan and 0.54 ml (5 mmol) of 2-fluorobenzonitrile to prepare 960 mg of the title compound. MS(ESI+) 226.1.

### Example 12

### 2-(3-Trifluoromethylphenyl)-3-amino-6-trifluoromethyl-benzo[b]thiophene

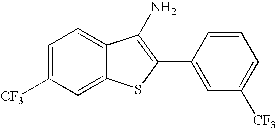

The procedure of Example 1 was repeated using 1.17 g (5.5 mmol) of 3-trifluoromethylbenzenemethanethiol and 1.08 g (5 mmol) of 2-nitro-4-(trifluoromethyl)benzonitrile. However, addition of water caused precipitation of product as a gum. The gum was extracted into ethyl acetate and the organic phase was washed with water then brine. The solution was dried using sodium sulfate then concentrated under vacuum to 2.30 g of an oil. The oil was crystallized by dissolution in hot isopropanol followed by addition of water at room temperature. Collection by filtration and drying in vacuum oven at 45° C. gave 1.87 g of yellow solid. NMR analysis confirmed the desired product contaminated with 3-trifluoromethylbenzenemethanethiol. Recrystallization from hot hexane afforded 690 mg the title product. MS(ESI+) 362.1.

### Example 13

### 2-phenyl-3-diazonium-6-benzyloxybenzo[b]thiophene tetrafluoroborate

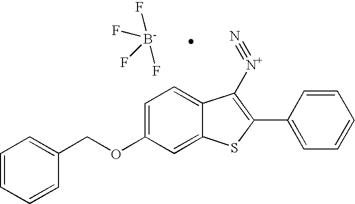

A 25 ml three neck round bottom flask fitted with a thermometer and magnetic stirrer was charged with 1.10 g (3.3 mmol) of 2-phenyl-3-amino-6-benzyloxybenzo[b]thiophene, 3.32 ml of water and 1.66 ml of 12M hydrochloric acid. The mixture was cooled to 0° C. and a solution of 0.35 g (5.0 mmol) of sodium nitrite in 0.5 ml of water was added over 5 minutes. The thick mixture was stirred at 0° C. for 30 minutes. A solution of 520 mg (4.7 mmol) of sodium tetrafluoroborate in 1.66 ml of water was added resulting in a very thick mixture which was difficult to stir. Addition of 10 ml of diethyl ether facilitated stirring. The mixture was stirred for 40 minutes and then filtered to collect the solid. The filter cake was washed 2 times with 4 ml of cold water, then 2 times with 10 ml of diethyl ether. The solid was dried in a vacuum oven at 30° C. for 3 hours. After drying, the solid was slurried in 10 ml of chloroform for 5 minutes, filtered and washed with 10 ml of chloroform 2 times. The solid was dried in a vacuum oven at 30° C. to give 1.05 g of the title compound. NMR analysis showed no amine and a downfield shift in the aromatic protons. IR (KBr pellet) analysis showed a strong peak at 2194 cm⁻¹, indicative of diazonium.

### Example 14

### 2-Phenyl-6-(benzyloxy)benzo[b]thiophene

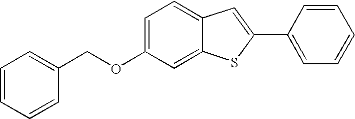

The diazonium tetrafluoroborate product (215 mg, 0.5 mmol) from Example 13 was added to a 25 ml three neck round bottom flask fitted with thermometer, reflux condenser, magnetic stirrer and heating mantle. THP (5 ml) was added and the contents were heated at reflux for 2 hours open to the atmosphere. The solution was cooled to room temperature and 5 ml of water was added. The resulting mixture was stirred for 1 hour, then filtered. The solid was washed with 10 ml of a 1:1 mixture of THF and water, then dried in vacuum oven at 45° C. to give 84.8 mg of the title compound. NMR analysis showed desired product, i.e., was identical to that of an authentic sample prepared via other well-known routes.

We claim:

1. A process for preparing a compound of formula I: 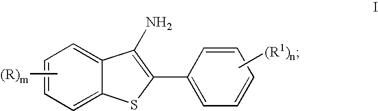
wherein:
m is 0 or 1;
n is an integer from 0 to 5;
R and R¹ are independently at each occurrence OH, NH₂, CF₃, CCl₃, CN, halo, C₁-C₆ alkoxy; C₃-C₇ cycloalkoxy, OC(O)(C₁-C₆ alkyl), OC(O)(C₃-C₇ cycloalkyl), OCO₂(C₁-C₆ alkyl), OCO₂(C₃-C₇ cycloalkyl), OSO₂(C₄-C₆ alkyl), OCON(R²)₂, OAr, OCOAr, OCO₂Ar, OCH₂Ar, OC(O)CH₂Ar, OCO₂CH₂Ar, OPO(C₁-C₆ alkyl)₂, OPO(Ar)₂, OPO(C₁-C₆ alkoxy)₂, OPO(OAr)₂, OCH₂NHC(O)R³, OCH₂OR³, OCH₂SR³;
R² is independently at each occurrence hydrogen, C₁-C₆ alkyl, C₃-C₇ cycloalkyl, or Ar;
R³ is C₁-C₆ alkyl or Ar; and
Ar is an optionally substituted phenyl group;
which includes reacting a compound of formula II: 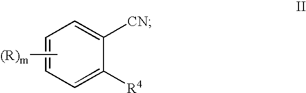
where R⁴ is fluoro or nitro; with a compound of formula III: 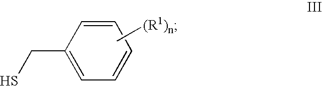
in the presence of a suitable polar aprotic solvent and between 1.01 and 3.0 equivalents, relative to the formula III compound, of a suitable kinetic base.

2. The process according to claim 1 wherein m and n are 1, R and R¹ are both methoxy, R is at the meta-position, relative to R⁴, of the phenyl ring and R¹ is at the para position.

3. The process according to claim 1 wherein m and n are 1, R is benzyloxy or isopropoxy, R¹ is methoxy, R is at the meta-position, relative to R⁴, of the phenyl ring and R¹ is at the para position.

4. The process of any one of claims 1-3 where the kinetic base is sodium hydride and between 2.0 and 3.0 molar equivalents of said base are employed

5. In a process for preparing a compound of formula XI: 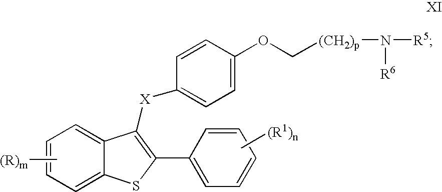
or an acid addition salt thereof; wherein:
p is 0, 1 or 2;
R⁵ and R⁶ are independently C₁-C₄ alkyl, or combine together with the nitrogen to which they are attached to form a piperidinyl, pyrrolidinyl, methylpyrrolidinyl, dimethylpyrrolidinyl, morpholino, dimethylamino, diethylamino, or 1-hexamethyleneimino ring; and
X is O or CO;
the improvement which comprises the process of claim 1.
